SEQUENCE LISTING (1 sequence)

SEQ ID NO: 1 - protein, 4 aa, Artificial Sequence
  synthesis
VHVV

(19) United States
(12) Patent Application Publication
(10) Pub. No.: US 2015/0368296 A1
(43) Pub. Date: Dec. 24, 2015

(54) Method for preventing or treating heart diseases by using a composition containing an isolated peptide

(71) Applicant: Tunghai University, Taichung (TW)

(72) Inventors: Wen-Dee Chiang, Taichung (TW); Wan-Teng Lin, Taichung (TW)

(21) Appl. No.: 14/593,967

(22) Filed: Jan. 9, 2015

(30) Foreign Application Priority Data
Jun. 24, 2014 (TW) 103121754

(51) Int. Cl. C07K 5/103

(52) U.S. Cl. CPC C07K 5/101; A61K 38/00

(57) ABSTRACT

This present invention discloses an isolated peptide which amino acid sequence is SEQ ID NO.:1. The isolated peptide can inhibit the expressions of signal transduction proteins related to the heart hypertrophy pathway, apoptosis of myocardial cells related proteins, and cardiac fibrosis related proteins to have effects of preventing such as apoptosis of myocardial cells, cardiac fibrosis, heart hypertrophy, cardiac inflammation or other heart diseases. Therefore, the peptide disclosed in this present invention can be an active ingredient of pharmaceutical composition to have effects for preventing or treating cardiovascular diseases.

The current application claims a foreign priority to the patent application of Taiwan No. 103121754 filed on Jun. 24, 2014.

### BACKGROUND OF THE INVENTION

1. Field of the Invention

This invention relates to the use of peptide, specially relates to a method for preventing or treating heart diseases by using a composition containing an isolated peptide.

2. Description of the Related Art

The cardiovascular diseases (CVD) include coronary heart disease (CHD), cerebrovascular disease, hypertensive heart disease, stroke, myocardial infarction, and atherosclerosis etc. According to statistic, about one-third population all over the world is dead because of CVD. Herein, there are over seventy million people in America carrying CVD or symptoms of it. In Taiwan, dead people from CVD are about one-fourth in all causes of death every year.

For example, coronary heart disease (CHD) is the most common heart disease and also major cause of sudden death. CHD mainly happens because of atherosclerosis which is resulted from the changing of the structure and composition of tunica intima to cause damage of blood flow function in blood vessels or insufficient of blood flow. When atherosclerosis happens on a heart coronary artery, it will trigger heart disease symptoms such as myocardial infraction or angina pectoris, and induce necrosis and apoptosis of myocardial cells.

Heart hypertrophy is one of physical symptoms accompanied by cardiovascular diseases in clinic, such as hypertrophy, angina pectoris, heart failure, stroke and artery diseases. Specifically speaking, the heart of adult mammals lack or almost have no regeneration ability, so the number of myocardial cells will not increase after born. When the heart under outer stimulations such as diseases and lacking oxygen, the myocardial cells will get bigger and become hypertrophy. Heart hypertrophy will proliferate fibers between myocardial cells or increase fibro tissues around coronary artery to induce fibrosis of myocardial cells or deficient of vessel function and trigger apoptosis of myocardial cells. When apoptosis of myocardial cells happens, the object will become a member of the high-risk group of heart failure and even dead when it becomes severe.

As we know that cardiovascular diseases have been a healthy problem all over the world, but treating or preventing cardiovascular diseases are getting difficult because of various causes of cardiovascular diseases such as heredity, smoking, diary, ages and hypertension. Now, giving an advice to change living and diary habit is the only way for an object who is the high-risk group of having coronary artery heart disease in clinic, there is no effective way to prevent it. β-adrenergic blockers or calcium channel blockers are used for patients having coronary artery heart diseases in the beginning to control it. Surgery treatment will perform if the situation is severe and the patients need to take anticoagulant drugs for long time after surgery. But clinic drugs for treating heart diseases have side effects like headache and heart beat fast. In another words, these drugs cannot be taken by patients for long time.

Therefore, the present method for treating heart diseases has highly side effects for patients to engage them into potential risks, and cannot provide a method for the high-risk group of heart diseases to prevent it.

### SUMMARY OF THE INVENTION

The major propose of this present invention is to provide a method for using an isolated peptide which can effectively treat or prevent cardiovascular diseases or related symptoms, especially heart hypertrophy, apoptosis of myocardial cells and cardiac fibrosis related diseases.

Another purpose of this present invention is to provide a method for preventing or treating heart diseases by using a composition containing an isolated peptide. It can decrease side effects of the delivered subject and increase absorptivity of the pharmaceutical composition on the delivered subject.

Further another purpose of this present invention is to provide a method for preventing or treating heart diseases by using a composition containing an isolated peptide which can be produced by easy biosynthesis for lowering the costs of manufacturing it and providing a stable quality and effectiveness.

In order to achieve these aforesaid purposes, this present invention discloses a method for treating or preventing cardiovascular diseases by delivering an effective amount of an isolated peptide to a subject, wherein the amino acid sequence of the isolated peptide is SEQ ID NO.: 1.

In one embodiment of this present invention, the cardiovascular diseases are related the diseases with symptoms such as heart hypertrophy, apoptosis of myocardial cells or cardiac fibrosis.

In the other embodiments of this present invention, it discloses that the composition can be a pharmaceutical composition or a nutritional supplement for preventing cardiovascular diseases.

### BRIEF DESCRIPTION OF THE DRAWINGS

FIG. 1 is the body weight diagram of mice from group 1 and 2.

FIG. 2 is the expressions of Bcl-2 protein, caspase-3 and cytochrome C in the heart tissues of mice from the each group.

FIG. 3A is the expression levels of Bcl-2 protein in the heart tissues of mice from the each group.

FIG. 3B is the expression levels of cytochrome C in the heart tissues of mice from the each group.

FIG. 3C is the expression levels of caspase-3 in the heart tissues of mice from the each group.

FIG. 4 is the expressions of nuclear factor of activated T-cells C3 (NFTA-C3) and calcineurin in the heart tissues of mice from the each group.

FIG. 5A is the expression levels of NFTA-C3 in the heart tissues of mice from the each group.

FIG. 5B is the expression levels of calcineurin in the heart tissues of mice from the each group.

FIG. 6 is the expressions of signal transducer and activator of transcription 3 (STAT-3), interleukin 6 (IL-6), mitogen-activated protein kinase kinase 5 (MEK-5) and extracellular signal-regulated kinase 5 (ERK-5) in the heart tissues of mice from the each group.

FIG. 7A is the expression levels of IL-6 in the heart tissues of mice from the each group.

FIG. 7B is the expression levels of STAT-3 in the heart tissues of mice from the each group.

FIG. 7C is the expression levels of MEK-5 in the heart tissues of mice from the each group.

FIG. 7D is the expression levels of ERK-5 in the heart tissues of mice from the each group.

FIG. 7E is the expression levels of active ERK-5 in the heart tissues of mice from the each group.

FIG. 8 is the expressions of p38α protein and Jun N-terminal kinase (JNK) in the heart tissues of mice from the each group.

FIG. 9A is the expression levels of p38α protein in the heart tissues of mice from the each group.

FIG. 9B is the expression levels of JNK in the heart tissues of mice from the each group.

FIG. 10 is the expressions of fibroblast growth factor 2 (FGF-2), extracellular signal-regulated kinase 1 (ERK-1), urokinase-type plasminogen activator (UPA), matrix metalloproteinase 2 (MMP-2) and matrix metalloproteinase 9 (MMP-9) in the heart tissues of mice from the each group.

FIG. 11A is the expression levels of FGF-2 in the heart tissues of mice from the each group.

FIG. 11B is the expression levels of ERK-1 in the heart tissues of mice from the each group.

FIG. 11C is the expression levels of UPA in the heart tissues of mice from the each group.

FIG. 11D is the expression levels of MMP-2 in the heart tissues of mice from the each group.

FIG. 11E is the expression levels of MMP-9 in the heart tissues of mice from the each group.

FIG. 12 is the expressions of atrial natriuretic peptide (ANP) and brain natriuretic peptide (BNP) in the heart tissues of mice from the each group.

FIG. 13A is the expression levels of ANP in the heart tissues of mice from the each group.

FIG. 13B is the expression levels of BNP in the heart tissues of mice from the each group.

FIGS. 14A to 14D are stained sections of the heart tissues of mice in sequence from the each group.

### DETAILED DESCRIPTION OF THE INVENTION

This present invention discloses an isolated peptide which amino acid sequence is SEQ ID NO.: 1. The isolated peptide can inhibit expression of signal transduction of heart hypertrophy pathway related proteins, apoptosis of myocardial cells related proteins, and cardiac fibrosis related proteins to have effects of preventing heart diseases such as apoptosis of myocardial cells, cardiac fibrosis, heart hypertrophy, cardiac inflammation and etc.

The isolated peptide which amino acid sequence is SEQ ID NO.: 1 disclosed in this present invention can be extracted from organisms, separated by hydrolysis or chemical synthesis. A person skilled in the art of this present invention and having general knowledge can understand and achieve the same effect by adding other extra modified peptide segments at the 5′ or 3′ end of amino acid sequence SEQ ID NO.: 1 without influence with normal physical function of the peptide to increase stability or feature of the peptide in this present invention.

The peptide disclosed in this present invention can be an effective ingredient of a pharmaceutical composition for preventing or treating cardiovascular diseases, wherein the pharmaceutical composition can be prepared into different form according to the delivering way, including but not limited to drop, powder, injection, pill, tablet, patch or oral etc.

The term “pharmaceutical composition” means that it includes an effective dosage of the peptide disclosed in this present invention and a pharmaceutically acceptable carrier, wherein the carrier including but not limited to water, alcohol, gel, glycerol, starch, mineral oil, vegetable oil, protein, diluter, excipient, suspension agent, antioxidant, stabilizer, colorant, perfume or filler, etc.

The peptide disclosed in this present invention can be an ingredient of a nutritional supplement for preventing cardiovascular diseases. By administering a determined dose of the nutritional supplement to a subject, it can achieve prevention of cardiovascular diseases or happening of related symptoms thereof.

Hereinafter, there are several examples and figures for further illustrating as followings.

The words mentioned in the specification of this present invention are illustrated as followings. But, the following explanations do not tend to limit the specification and claims of this present invention. The words without explanations are defined according to the meanings understood by a person skilled in the art of this present invention and having general knowledge.

The term “Bcl-2 protein” means the B-cell lymphoma-2 protein which is related to the signal transduction pathway of apoptosis. Because Bcl-2 protein can attach to mitochondria for maintaining the balance of it and reducing release of cytochrome C, further to achieve the effect of inhibiting apoptosis. Therefore, the Bcl-2 protein is categorized as an inhibitor of apoptosis protein.

The term “calcineurin/nuclear factor of activated T-cells pathway” is considered related to heart hypertrophy, apoptosis and survival of myocardial cells. Specifically speaking, when a myocardial cell receives outside stimulations, the concentration of calcium in the myocardial cell will increase to change the structure of calcineurin and activate it. Activated calcineurin can catalyze downstream nuclear factor of activated T-cells and the calcineurin cuts the phosphate of C family of nuclear factor of activated T-cells after combing with the C family of nuclear factor of activated T-cells. The transcription in the nucleus of cell will be affected and lead to symptoms of heart hypertrophy.

### Example 1

### Preparing the Animal Model of Heart Hypertrophy

This example was proceeded under the animal experiment protocol (IAUCUC-100-12) approved by the ethic committee of Academia Sinica Institutional Animal Care and Utilization Committee (IACUC).

The male C57BL/6 mice at the age of 6 weeks were divided into 5 groups for 8 mice in each group. The each group of mice were feed at 24±2° C. of temperature, humidity of 55±10% and 12/12 light cycle for 8 weeks, wherein the group 1 was control group on a normal diet (PMI Nutrition International, Brentwood, Mo., USA) and injected 0.9% saline intraperitoneally; the group 2 was fed high-fat diet to induce the mice into heart hypertrophy model and injected 0.9% saline intraperitoneally; the group 3 was fed high-fat diet and SEQ ID NO.:1 peptide was injected intraperitoneally with the dose of 5 mg/kg/day; the group 4 was fed high-fat diet and SEQ ID NO.:1 peptide was injected intraperitoneally with the dose of 10 mg/kg/day; the group 5 was fed high-fat diet and SEQ ID NO.:1 peptide was injected intraperitoneally with the dose of 25 mg/kg/day. The injection period of each group of mice was between the third week to the sixth week of feeding period. The high-fat diet contained 60% of energy as fat.

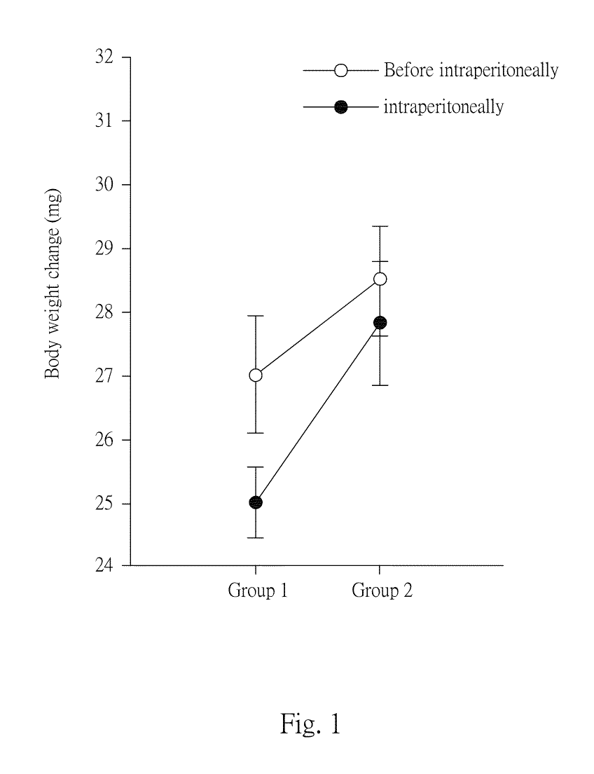

The body weights of the groups 1 and 2 were record during feeding and the result was shown at FIG. 1 after statistically proceeding. FIG. 1 illustrated that the body weight of the mice from the group 2 were obviously higher than that of the mice from the group 1. It shows that the feeding the high-fat diet can induce the mice obesity.

After the feeding period, blood of the each group of mice were collected and then the each group of mice were sacrificed for using of the following examples.

### Example 2

### Extraction of Heart Tissue Protein

The mice heart tissues from the each group of example 1 were put into the lysis buffer (100 mg/ml) and homogenized. Then, the homogenates of the mice from the each group were placed on ice, centrifuged at 12000 g for about 40 minutes. The supernatants were collected and stored at −80° C. for further uses.

### Example 3

### Western Blotting

Protein concentration of the extract was determined by Lowry's protein assay method. A proteins sample was separated in a 12% SDS-PAGE with 75V constant power supply. The proteins were then transferred to the PVDF membrane (GE Healthcare Life Science Co., USA) using 50 V current for 3 hours. The transferred membrane was incubated in Tris buffer saline (TBS) with 3% bovine serum albumin and then added a predetermined primary antibody onto the PVDF membrane for conjugation with specific proteins. Horseradish peroxidase-labeled secondary antibodies were used for detection and pictures were finally taken with Fujifilm LAS-3000 (GE Healthcare Life Sciences). Primary antibodies against a-tubulin (Oncogene Science, Inc., Uniondale, N.Y., USA), Bcl-2 (Transduction Laboratories, Lexington, Ky., USA), cytochrome C (BD Pharmingen, San Diego, Calif., USA), cleaved caspase-3, matrix metalloproteinase-9 (MMP-9), poly (ADP-ribose) polymerase (PARP), RhoA (Cell Signaling Technology, Inc., Beverly, Mass., USA), p-ERK5, ERK5 (Upstate/Millipore, Billerica, Mass., USA), and ANP (Abcam, Cambridge, Mass., USA) were used. All other antibodies were from Santa Cruz Biotechnology (Santa Cruz, Calif., USA).

### Example 4

### The Effect of the Peptide from this Present Invention to the Related Apoptosis Regulatory Proteins

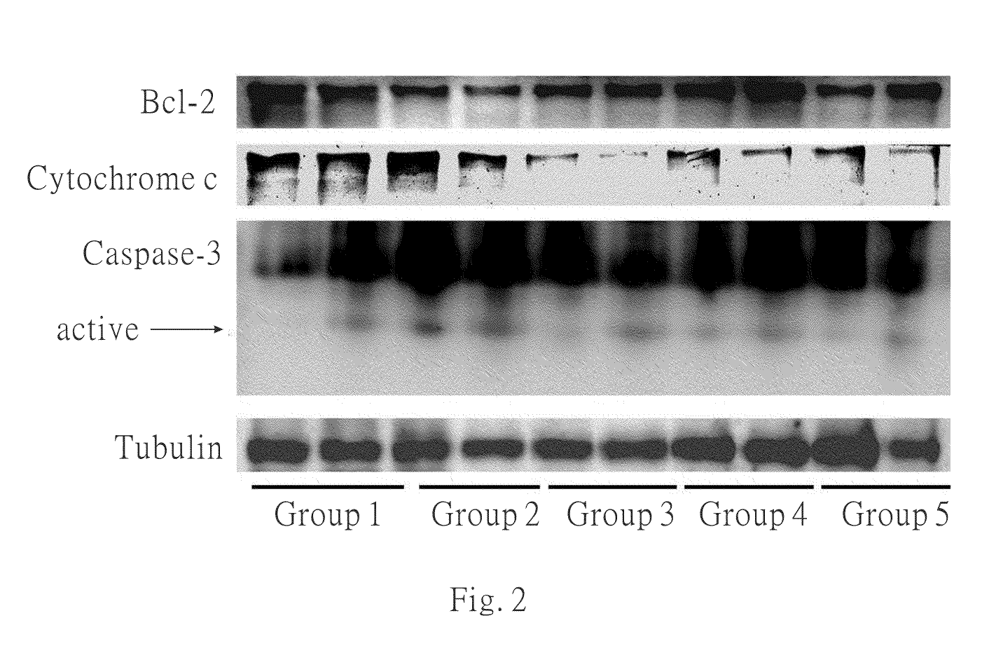

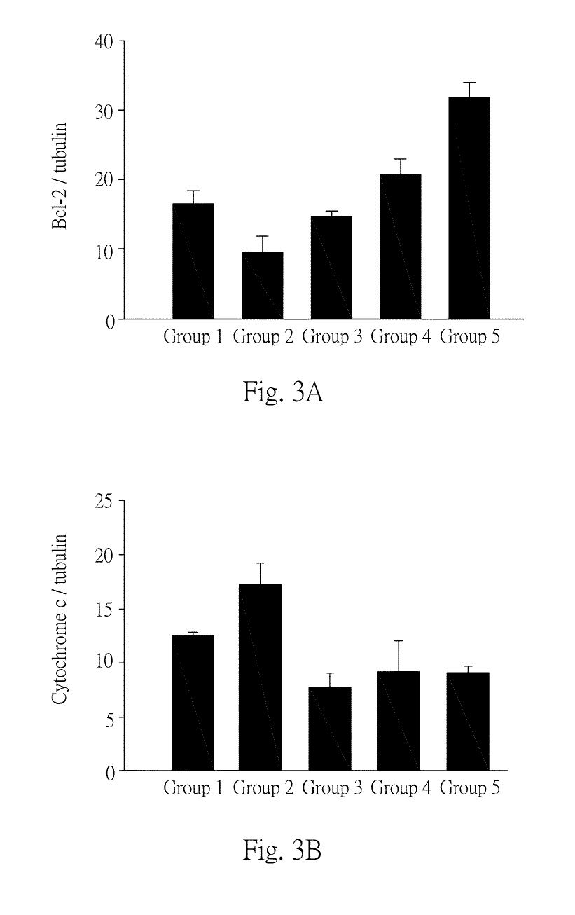

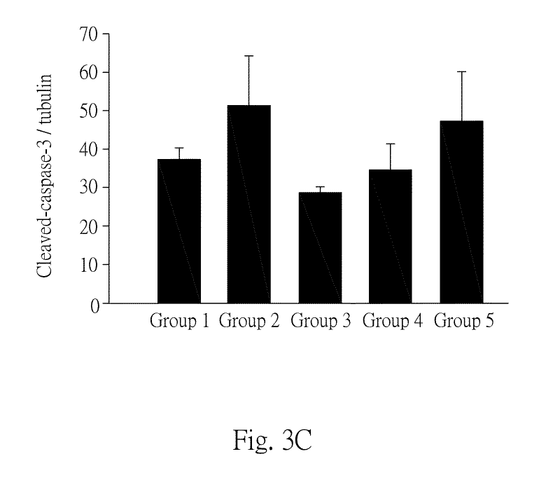

The protein extraction of the mice heart tissues from the each group in example 2 were processed by the western blot method in example 3 to observe the expressions of related apoptosis regulatory proteins in the mice heart tissues from the each group. The results were shown at FIGS. 2 and 3, wherein the tubulin was used for an internal control in FIG. 2 and the expression levels of related apoptosis regulatory proteins were calculated basing on the expression level of tubulin in each figure of FIG. 3.

Results from FIGS. 2 and 3 illustrated that compared with the group 1, the expression level of Bcl-2 in the heart cell of the mice from the group 2 was reduced and the expression level of active caspase-3 of the mice from the group 2 was increased according to the increased expression level of cytochrome C. The expression levels of Bcl-2 in the heart cell of the mice from the groups 3 to 5 were obviously increased in comparison of the mice from the group 2, respectively. The expression levels of cytochrome C and activated caspase-3 in the heart cell of the mice from the groups 3 to 5 were obviously decreased in comparison of the mice from the group 2, respectively.

The results of FIGS. 2 and 3 illustrate that high-fat diet will decrease the expression level of Bcl-2 protein in heart tissues and stability of the mitochondria membrane will be affected to release cytochrome C and further activate caspase-3. Activated caspase-3 affects downstream proteins to lead apoptosis of myocardial cells. The subject under high-fat diet will obviously increase the expression level of Bcl-2 in the heart tissues by administering the peptide having amino acid sequence in SEQ ID NO.: 1 from this present invention and decrease the expression level of cytochrome C for inhibiting the activation of caspase-3. Therefore, the peptide having amino acid sequence in SEQ ID NO.: 1 from this present invention can effectively inhibit apoptosis of heart cells to achieve the effect of treating or preventing cardiovascular diseases.

### Example 5

### The Effect of the Peptide from this Present Invention to the Calcineurin/NFTA-C3 Pathway

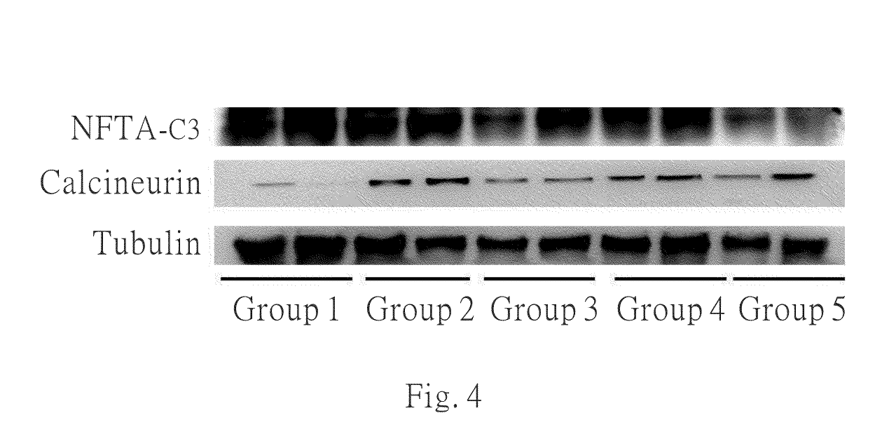

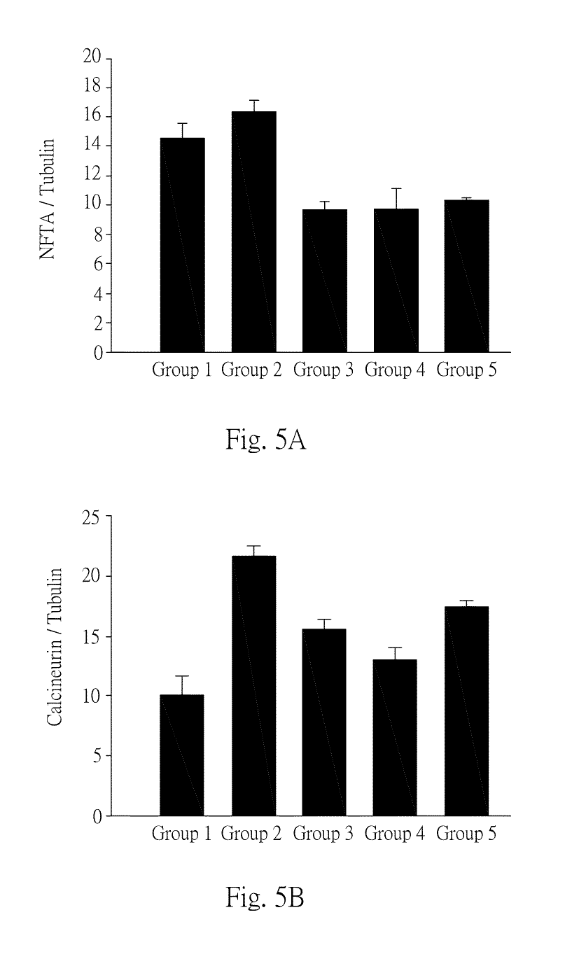

The protein extraction of the mice heart tissues from the each group in example 2 were processed by the western blot method in example 3 to observe the expressions of nuclear factor of activated T-cells C3 (NFTA-C3) and calcineurin in the mice heart tissues from the each group. The results were shown at FIGS. 4 and 5, wherein the tubulin was used for an internal control in FIG. 4 and the expression levels of NFTA-C3 and calcineurin were calculated basing on the expression level of tubulin in each figure of FIG. 5.

Results from FIGS. 4 and 5 illustrated that the expression levels of NFTA-C3 and calcineurin in the heart cells of the mice from the group 2 was significantly increased in comparison of the mice from the group 1. The expression levels of NFTA-C3 and calcineurin in the heart cells of the mice from the groups 3 to 5 were obviously decreased in comparison of the mice from the group 2, respectively.

According to the results of FIGS. 4 and 5, it reveals that the mice fed high-fat diet will actually activate the calcineurin/NFTA-C3 pathway to induce heart hypertrophy and apoptosis of myocardial cells. On the other hand, the mice fed high-fat diet can obviously inhibit the expression levels of NFTA-C3 and calcineurin in the heart tissues by administering the peptide having amino acid sequence in SEQ ID NO.: 1 from this present invention to stop the activation of the calcineurin/NFTA-C3 pathway.

Therefore, the peptide having amino acid sequence in SEQ ID NO.: 1 from this present invention can effectively inhibit heart hypertrophy and apoptosis of myocardial cells to achieve the effect of treating or preventing cardiovascular diseases.

### Example 6

### The Effect of the Peptide from this Present Invention to the Related Signal Transduction Proteins of Heart Hypertrophy

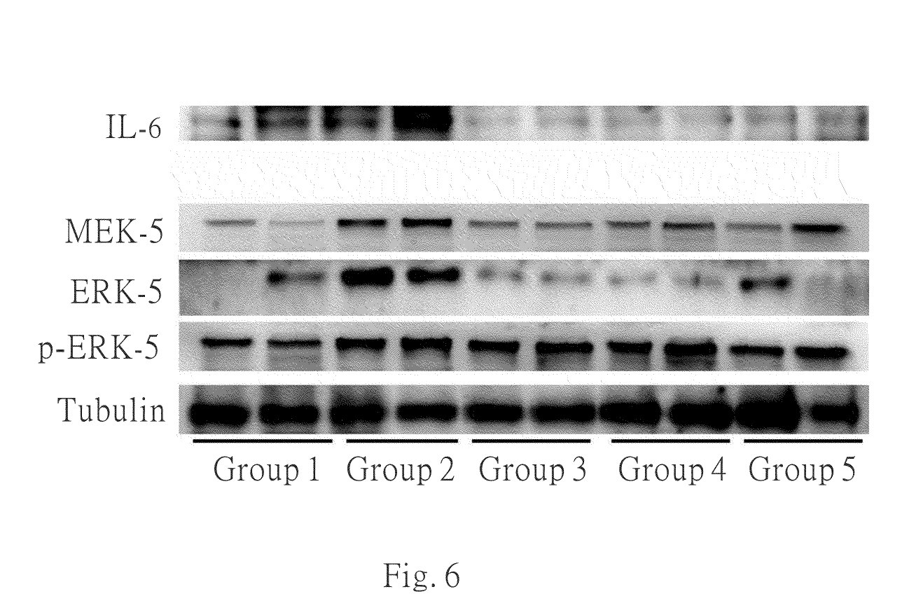

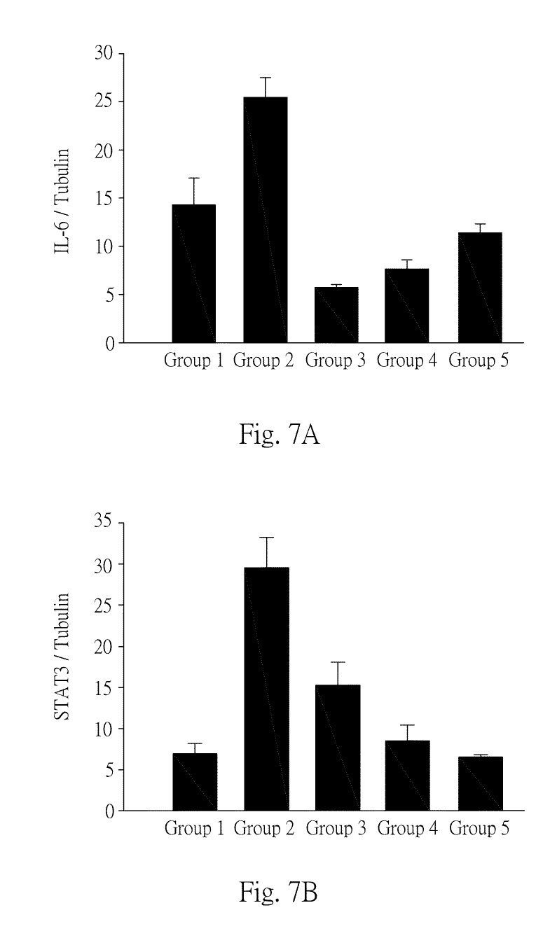

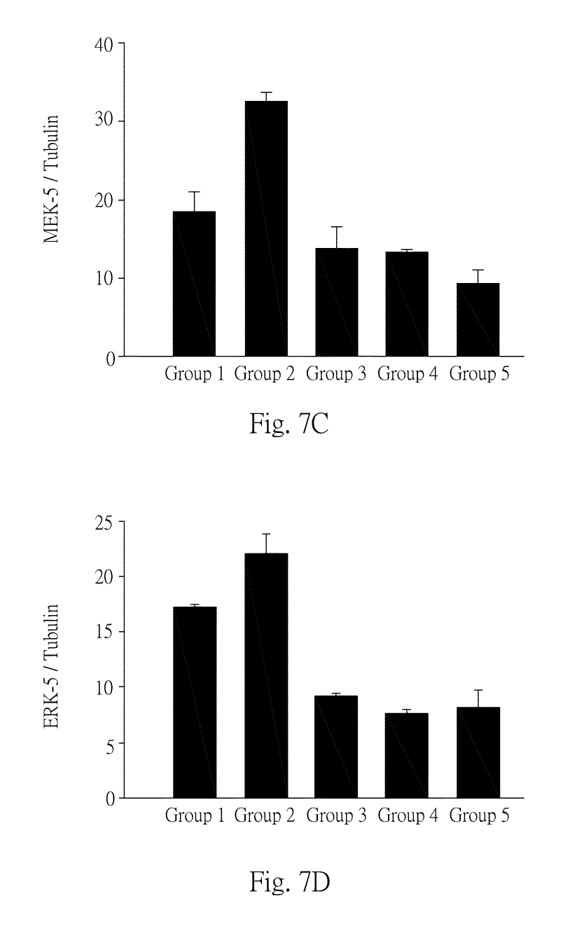

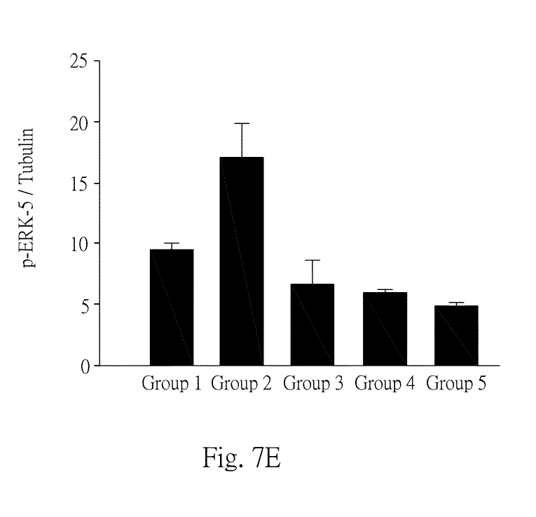

The protein extraction of the mice heart tissues from the each group in example 2 were processed by the western blot method in example 3 to observe the expressions of the related signal transduction proteins of heart hypertrophy including: STAT-3, IL-6, MEK-5 and ERK-5. The results were shown at FIGS. 6 and 7, wherein the tubulin was used for an internal control in FIG. 6 and the expression levels of the related signal transduction proteins of heart hypertrophy in the mice heart tissues from each group was calculated basing on the expression level of tubulin in FIG. 7.

Results from FIGS. 6 and 7 showed that the amount of the related signal transduction proteins of heart hypertrophy in the mice heart tissues of the group 2 was significantly higher than that of the group 1. The expression levels of the related signal transduction proteins of heart hypertrophy in the heart tissues of the mice from the groups 3 to 5 were obviously lower than that of the mice from the group 2. Therefore, aforesaid results indicate that the high-fat diet will indeed increase the expression levels of IL-6, activate STAT-3 and induce MEK-5/ERK-5 pathway to trigger symptoms of heart hypertrophy of mice. Otherwise, the mice having the high-fat diet can inhibit the expression of IL-6 to regulate the related pathway of heart hypertrophy from being activated, such as the STAT-3 pathway, MEK-5/ERK-5 pathway to avoid the heart hypertrophy.

Therefore, the peptide having amino acid sequence in SEQ ID NO.: 1 from this present invention has the effect of treating or preventing cardiovascular diseases.

### Example 7

### The Effect of the Peptide from this Present Invention to the Mitogen-Activated Protein Kinase (MAPKs) Signal Transduction Pathway

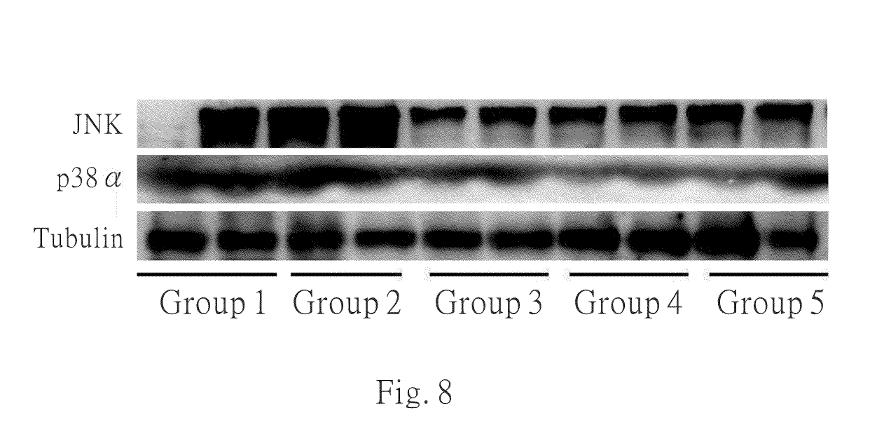

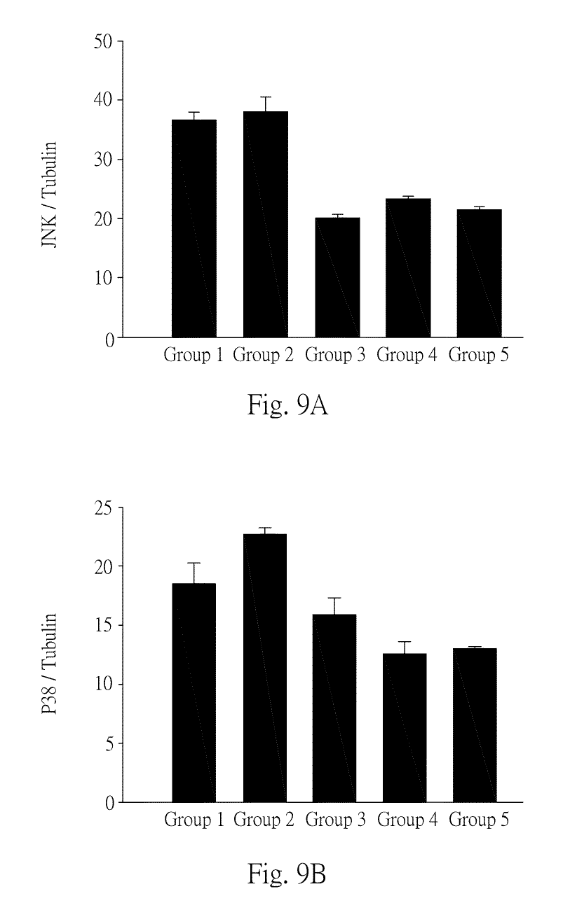

The protein extraction of the mice heart tissues from the each group in example 2 were processed by the western blot method in example 3 to observe the expressions of p38α protein and JNK. The results were shown at FIGS. 8 and 9, wherein the tubulin was used for an internal control in FIG. 8 and the expression levels of p38α protein and JNK were calculated basing on the expression level of tubulin in each figure of FIG. 9.

Results from FIGS. 8 and 9 illustrated that the expression levels of p38α protein and JNK in the heart cells of the mice from the groups 3 to 5 were significantly lower than the mice from the group 2, respectively. It shows that the activation of the p38α protein/JNK pathway will be inhibited by administering the peptide having amino acid sequence in SEQ ID NO.:1 from this present invention to avoid the related diseases having symptoms such as apoptosis of heart cells and heart inflammation.

Therefore, the peptide having amino acid sequence in SEQ ID NO.: 1 from this present invention has the effect of treating or preventing the cardiovascular diseases.

### Example 8

### The Effect of the Peptide from this Present Invention to the Cardiac Fibrosis

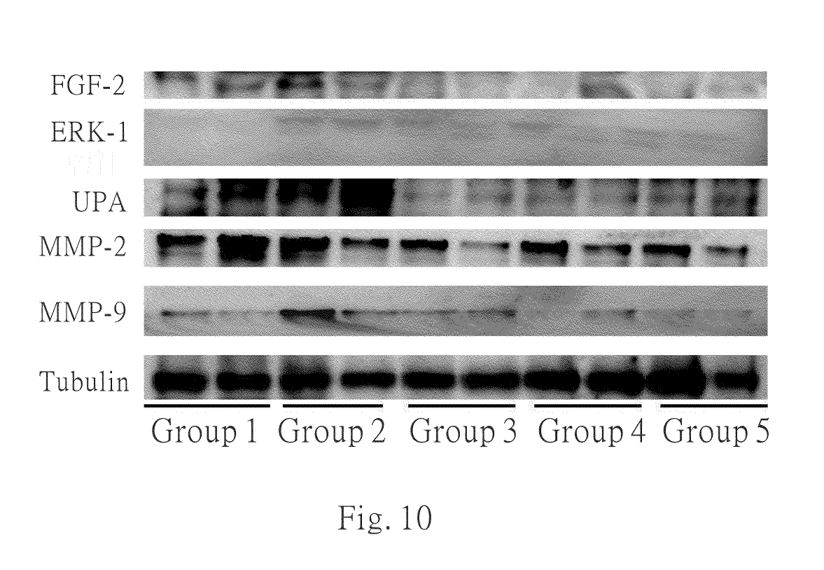

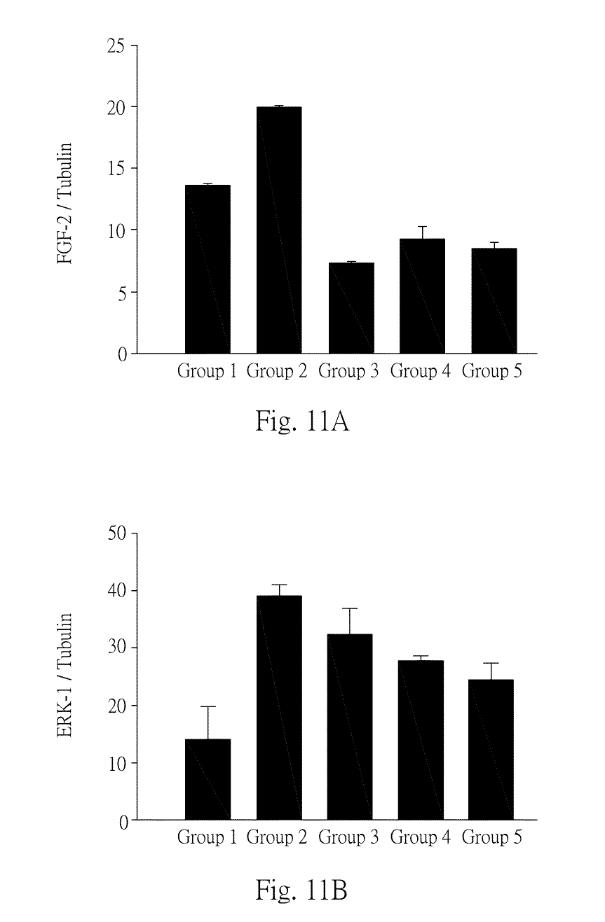

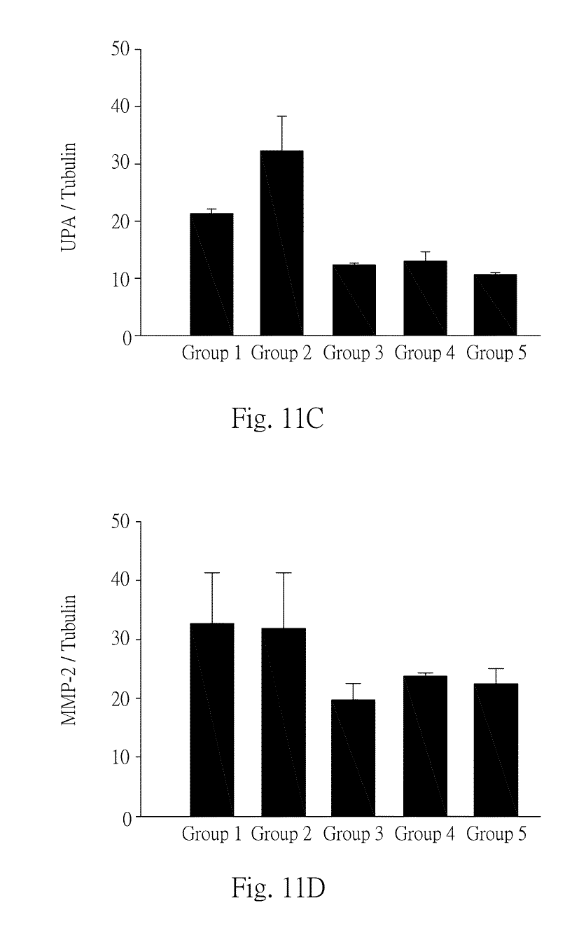

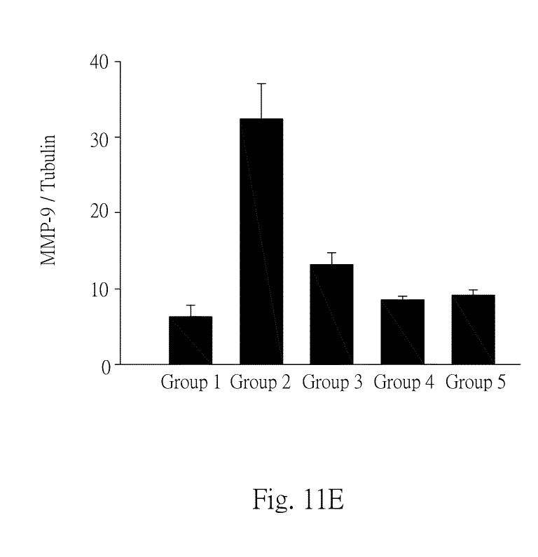

The protein extraction of the mice heart tissues from the each group in example 2 were processed by the western blot method in example 3 to observe the expressions of the related proteins of cardiac fibrosis pathway including: FGF-2, ERK-1, UPA, MMP-2 and MMP-9. The results were shown at FIGS. 10 and 11, wherein the tubulin was used for an internal control in FIG. 10 and the expression levels of FGF-2, ERK-1, UPA, MMP-2 and MMP-9 in the mice heart tissues from the each group was calculated basing on the expression level of tubulin in each figures of FIG. 11.

Results shown at FIGS. 10 and 11 revealed that the related proteins of cardiac fibrosis pathway in mice heart tissues from the groups 3 to 5 were obviously lower than the mice of the group 2, respectively. It illustrates that the expressions of the related proteins of cardiac fibrosis pathway can be inhibited by administering the peptide having amino acid sequence in SEQ ID NO.: 1 from this present invention to decrease the risk of cardiac fibrosis in the mice fed the high-fat diet. Therefore, administering the peptide having amino acid sequence in SEQ ID NO.:1 from this present invention can inhibit the activation of cardiac fibrosis pathway to achieve the effect of treating or preventing cardiovascular diseases.

### Example 9

### The Effect of the Peptide from this Present Invention to the Heart Hypertrophy

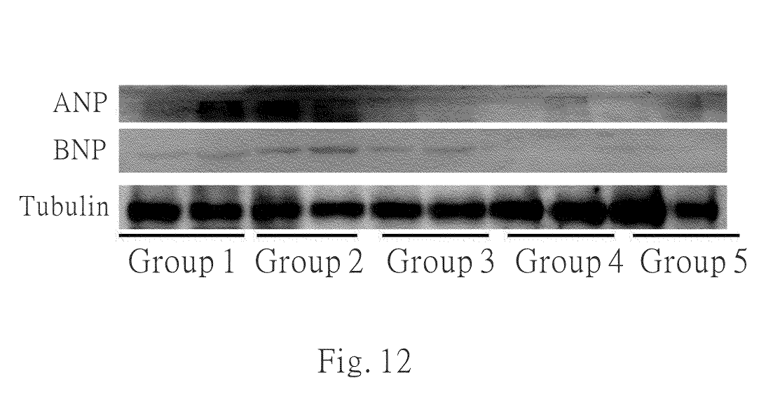

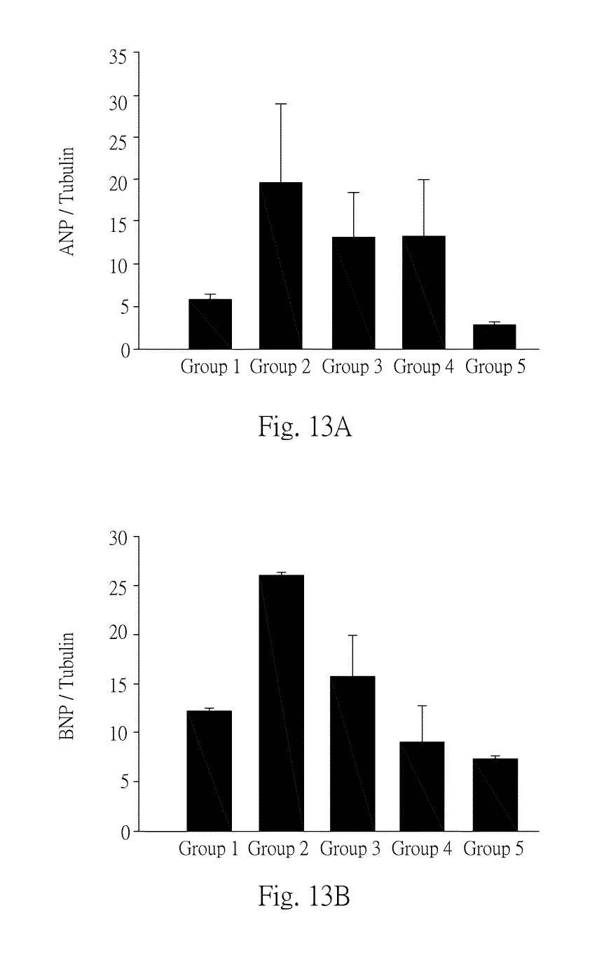

The protein extraction of the mice heart tissues from the each group in example 2 were processed by the western blot method in example 3 to observe the expressions of the indicator of heart hypertrophy and heart failure. The results were shown at FIGS. 12 and 13, wherein the tubulin was used for an internal control in FIG. 12 and the expression levels of the ANP and BNP in the mice heart tissues from the each group was calculated basing on the expression level of tubulin in each figures of FIG. 13.

Results from FIGS. 12 and 13 illustrated that the expression levels of ANP and BNP in the mice heart tissues of the group 2 was significantly higher than the mice from the group 1. The expression levels of ANP and BNP in the heart tissues of the mice from the groups 3 to 5 were obviously decreased in comparison of the mice from the group 2, respectively.

Specifically speaking, the mice fed the high-fat diet will largely express ANP and BNP. It reveals that the high-fat diet will not only lead to heart hypertrophy but also faster induction of heart failure. The mice fed the high-fat diet can effectively decrease the expression levels of ANP and BNP in the heart tissues. Therefore, the peptide having amino acid sequence in SEQ ID NO.: 1 from this present invention can improve the symptoms of heart hypertrophy or heart failure to achieve the effect of treating or preventing cardiovascular diseases.

### Example 10

### Stained Section of Heart Tissues

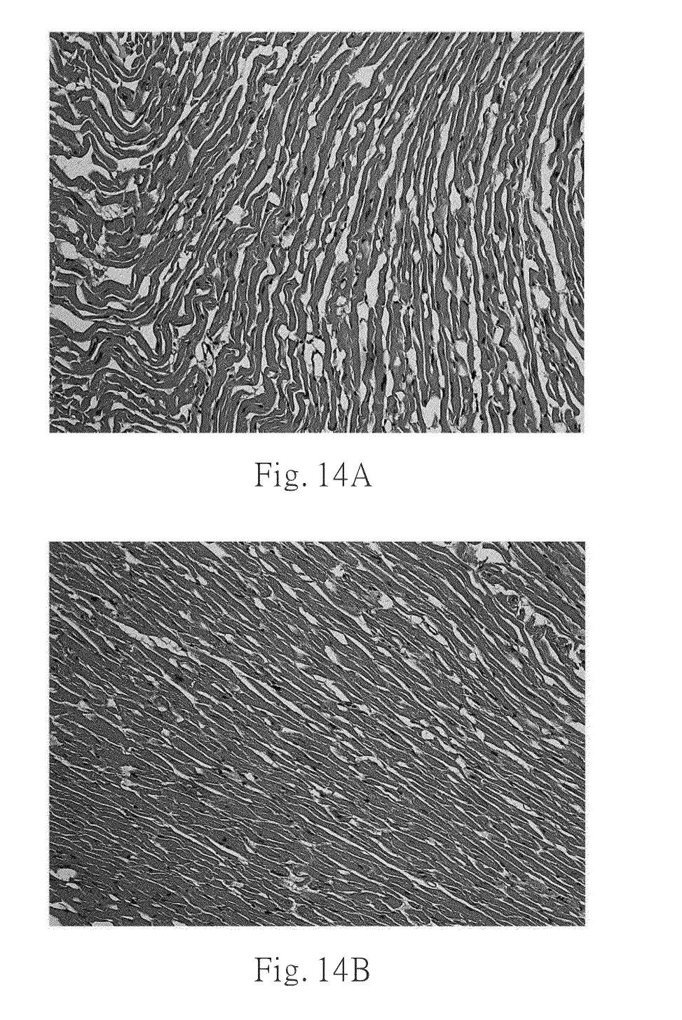

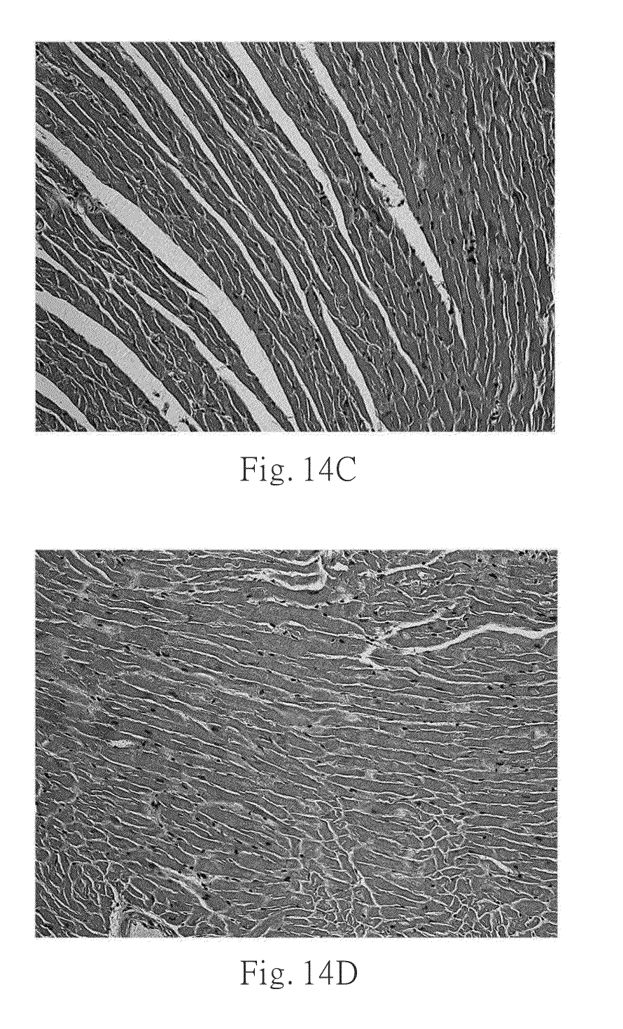

The feeding method was the same with example 1. Mice were divided into 4 groups for feeding at different conditions. The intraperitoneal injection was performed between the third week and the sixth week of the feeding period. Wherein, the group 1 was control group fed a normal diet and injected 0.9% saline intraperitoneally; the group 2 was fed the high-fat diet to induce the mice into heart hypertrophy model and injected 0.9% saline intraperitoneally; the group 3 was fed the high-fat diet and injected SEQ ID NO.:1 peptide intraperitoneally with the dose of 15 mg/kg/day; the group 4 was fed the high-fat diet and injected SEQ ID NO.:1 peptide intraperitoneally with the dose of 25 mg/kg/day. Mice of groups 1 to 4 were all sacrificed and the heart tissues were taken and sliced after embedded into paraffin. After removing the paraffin, the sections of the mice from the each group were stained by H&E method and washed by water. The stained sections of the mice from the each group were observed by microscope and the results were shown at FIG. 14A to 14D.

FIG. 14 showed that myocardial cells of mice from the group 2 were deformed and disorderly arranged to indicate the blooming of cardiac fibrosis. In comparison with the mice from the group 2, the arrangement of myocardial cells of groups 3 and 4 were more orderly and closely and has normal structure of heart muscles. Therefore, the peptide having amino acid sequence in SEQ ID NO.: 1 from this present invention can effectively reduce or prevent the situation of the damages of heart tissues to achieve the effect of treating or preventing related heart diseases.

According to the aforesaid results, the peptide having amino acid sequence in SEQ ID NO.: 1 from this present invention indeed can inhibit signal transduction proteins of heart hypertrophy pathway, inhibit the cardiac fibrosis pathway and decrease apoptosis of myocardial cells for effectively improving or releasing symptoms of heart hypertrophy, cardiac fibrosis, inflammation of heart or heart failure. Therefore, the peptide having amino acid sequence in SEQ ID NO.: 1 from this present invention indeed has the effect of treating or preventing cardiovascular diseases.

It should be understood that the above-mentioned detailed description and specific examples are given by way of illustration only. Any easy changes or modifications base on examples in the description will be included within the scope of following claims.

What is claimed is:

1. A method for treating or/and preventing cardiovascular diseases, comprising administering a composition containing an effective amount of an isolated peptide to a subject, wherein the isolated peptide having amino acid sequence in SEQ ID NO.: 1.

2. The method according to claim 1, wherein the cardiovascular disease is the heart hypertrophy related diseases.

3. The method according to claim 2, wherein the cardiovascular disease is the apoptosis of myocardial cells related diseases.

4. The method according to claim 3, wherein the cardiovascular disease is the cardiac fibrosis related diseases.

5. The method according to claim 1, wherein the composition is a pharmaceutical composition.

6. The method according to claim 1, wherein the composition is a nutritional supplement.
